(19) United States
(12) Patent Application Publication
(10) Pub. No.: US 2007/0038474 A1
(43) Pub. Date: Feb. 15, 2007

(54) Workflow and communications logging functions of an automated medical case management system

(76) Inventor: Mark J. Halsted, Wyoming, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 11/501,468

(22) Filed: Aug. 9, 2006

Related U.S. Application Data
(60) Provisional application No. 60/706,625, filed on Aug. 9, 2005.

(51) Int. Cl. G06Q 10/00; G06F 19/00; G06Q 50/00; A61B 5/00

(52) U.S. Cl. 705/2; 705/3

(57) ABSTRACT

Exemplary embodiments of the present invention perform workflow and communications logging functions in a hospital radiology department, free-standing imaging center, or other institution providing imaging services. The exemplary embodiments allow logging of communications and attempted communications both within the department and with personnel outside the department. The logs permanently memorialize the communications and can be viewed with the patient's other computerized records. The exemplary embodiments also assign and tracks the status of work as it progresses through a multi-step process. The exemplary embodiments generate and update lists of assigned tasks for the various individuals involved in providing radiology services to patients. For radiologists, the task lists are created by filtering the lists of cases available for interpretation. The lists are updated to reflect the current pending cases in real time. Users can query the system to determine the exact status of any case. Additionally, the system permits computer-based communications to accompany radiological studies throughout the process.

### CROSS REFERENCE TO RELATED APPLICATIONS

The present application claims the benefit of U.S. Provisional Application Ser. No. 60/706,625 filed Aug. 9, 2005, the entire contents of which are incorporated herein by reference.

### BACKGROUND

Exemplary embodiments of the present invention provide a system and method for streamlining the flow of work and communications between personnel in a hospital radiology department. Additionally, exemplary embodiments of the present invention permanently memorialize communications and attempted communications between the various personnel involved in providing care for patients requiring the services of a radiology department, for example.

Conventionally, radiologists in a central “reading room” interpret exams received in a first-in first-served basis. For various reasons, the radiologists are often interrupted with requests for expedited examination of certain cases and/or requests for status on cases that are being examined or have yet to be examined. This drastically reduces the efficiency of the examination process and increases the stress level of the radiologists and all others involved in the process. Additionally, prior art systems often lack the capability to conveniently and permanently log communications both within the department and with outside personnel.

### SUMMARY

The present application is designed to complement the application disclosed in. co-pending PCT application, Ser. No. PCT US06/10660, filed Mar. 23, 2006, the disclosure of which is incorporated herein by reference. The co-pending application describes an “Automated System and Method for Prioritization of Waiting Patients” also known as the Automated Radiology Triage System (“ARTS”) or “RadStream.” Exemplary embodiments of the present invention function as a part of the larger ARTS system and utilizes the output of the patient prioritization algorithm and process found in the co-pending application.

Exemplary embodiments of the present invention provide a computerized communications, logging, and workflow system that increases the efficiency of patient care in a hospital radiology department. The system includes a graphical user interface (“GUI”) that allows users to communicate within the radiology department, coordinate communications with personnel outside the department, and permanently record all communications, attempted communications, and reports. In conjunction with the ARTS patient prioritization system described in the copending application mentioned above, the exemplary embodiments of the present invention generate task lists for individuals involved in the process of obtaining, interpreting and reporting the results of radiological examinations. Additionally, exemplary embodiments of the present invention allow users to search the computerized records by various criteria to determine the real-time status of a case.

In a first aspect of the present invention, the system facilitates communications between personnel involved in providing radiology services to patients. The system provides a substantially automated mechanism for enabling electronic communications between personnel within the radiology department and utilizes a computerized process to coordinate communications with personnel outside the radiology department.

In a second aspect of the present invention, the system permanently memorializes/stores communications (i.e., keeps an electronic record of such communications that cannot be deleted, if at all, without specific access/deletion rights) and attempted communications. All memorialized electronic communications and reports are time stamped upon entry into the system. When a member of the radiology department staff utilizes the system to log a communication with outside personnel, that log entry is also time/date stamped. All communications data collected by the system is permanently stored and can be readily retrieved (by a user with proper access rights) using built in search functions.

It is a third aspect of the invention to generate computerized task lists for personnel involved in providing radiology services to patients. Through an interface with the Radiology Information System (“RIS”), ARTS obtains data pertaining to all outstanding ordered examinations as well as the associated patient data. ARTS prioritizes the patients using the algorithm described in the abovementioned co-pending application. The present invention displays the prioritized patient/exam list for each member of the radiology staff in the form of a worklist. The worklists are constantly updated as cases move through the process.

In a fourth aspect of the invention, the system allows users to determine the real-time status of an examination. Any user can search for a case by various criteria and determine its status. This function allows any user questioned by a patient or physician, for example, to ascertain and report the status of an examination without multiple phone calls and without speaking to the personnel performing each step of the process. Other aspects and advantages will be apparent from the following detailed description, the attached drawings and the appended claims.

### BRIEF DESCRIPTION OF THE DRAWINGS

FIG. 1 shows a diagram of the ARTS system networked to a number of user computers.

FIG. 2 shows a conceptual web site diagram for the ARTS system.

FIG. 3 shows an exemplary log-in page used to by a user to log into the ARTS system.

FIG. 4 shows an exemplary shot of a screen used by a technologist to view a worklist.

FIG. 5 shows another exemplary shot of a screen used by a technologist to view a worklist.

FIG. 6 shows an exemplary shot of a screen used by a technologist to input acuity data for a particular patient and particular exam.

FIG. 7 shows an exemplary MD contact information pop-up window that may be used to designate additional physicians to receive information regarding a particular patient, exam, report.

FIG. 8 shows an exemplary shot of a screen that may be used by an optional reading room assistant to assign particular exams to particular radiologist on duty.

FIG. 9 shows an exemplary shot of a screen used by radiologists to designate the location at which exams will be read and choose the types of exams that will be read.

FIGS. 10a and 10b show an exemplary shots of a screen used by radiologists to view the radiologist's worklist.

FIG. 11 shows an exemplary communication tool.

FIG. 12 shows an exemplary shot of a screen used by a physician priority link (“PPL”) operator to view the PPL operator's worklist.

FIG. 13 shows an exemplary case exam information popup used by a PPL operator to view exam information.

FIG. 14 shows an exemplary shot of a screen used by a PPL operator to convey exam information to a physician contact.

FIG. 15 shows an exemplary shot of a screen used by a PPL operator to view the PPL operator's worklist and cases on hold.

FIG. 16 shows an exemplary radiologist worklist screen with an exemplary window showing exams waiting to be conveyed to a physician contact.

FIG. 17a shows an exemplary shot of a screen used to view conveyance records for a plurality of PPL operators.

FIG. 17b shows an exemplary shot of a screen used to view conveyance records for a single PPL operator.

FIG. 18 shows an exemplary shot of a screen used to search exam records.

FIG. 19 shows an exemplary shot of a screen used to view the results of an exam records search based on date restrictions.

FIG. 20 shows an exemplary shot of a screen used to view the results of an exam records search based on Radiologist name and date restrictions.

FIG. 21 shows an exemplary shot of a screen used to view the record details for a particular exam.

FIG. 22a shows an exemplary shot of a screen used to view patient information.

FIG. 22b shows an exemplary shot of a screen used to view exam information.

FIG. 22c shows an exemplary shot of a screen used to view acuity information.

FIG. 23 shows an exemplary shot of a screen used to view a contact log.

### DETAILED DESCRIPTION

Exemplary embodiments described herein pertain to workflow and communications functions supplementing the existing ARTS system, which pertains to a computerized system and method for prioritizing radiology examinations. While the exemplary embodiments described herein pertain to workflow and communications in a radiology context, it will be apparent to those of ordinary skill in the art that the invention may be used in other areas of medicine; and may also be used in areas beyond the medical field.

The process in which a radiology examination is obtained, interpreted, reported, and conveyed requires several personnel, each potentially at a different physical location, to perform independent finctions in a certain order and in a timely manner. The process generally begins with a radiology technologist obtaining a study. The technologist may add comments to accompany the study. The study is passed to a reading room where a reading room assistant assigns incoming studies to radiologists. A radiologist interprets the study and records a report of the results of the examination. The radiologist either passes the report back to the reading room assistant, for reports to be conveyed to ordering physicians within the hospital, or passes it to the “Physician Priority Link” (“PPL”), a bank of telephone operators who convey reports to outside ordering physicians. The reading room assistant or PPL operator communicates the report to the ordering physician.

The present invention provides a computerized communications, logging, and workflow system that increases the efficiency of the process described above. Specifically, the present invention facilitates communications (both internal and external to the radiology department), permanently memorializes communications and reports, generates constantly updated worklists, and provides a search function. For the purposes of the present application, “permanently” memorializing or storing an electronic record involves keeping an electronic record that typically cannot be deleted, if at all, without specific access/deletion rights

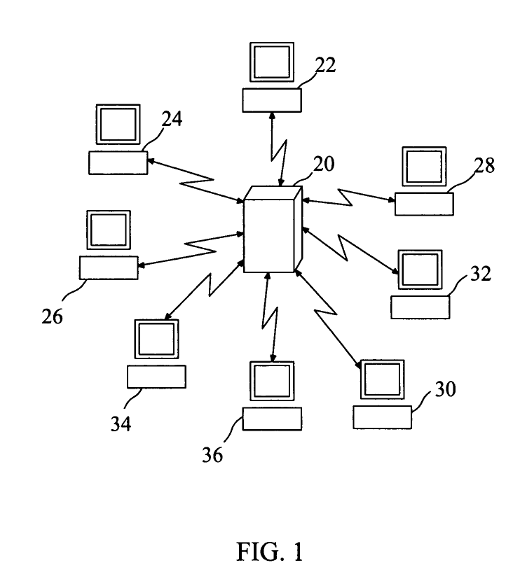

As shown in FIG. 1, ARTS exists as a software tool residing on a central server 20, which may be accessed by one or more workstations operatively coupled to the central computer through a direct connection or a network connection (wired or wireless). For example, in the exemplary embodiment, the ARTS tool is a web-based application accessible by the plurality of workstations over the internet. In the exemplary embodiment, the plurality of workstations accessing the ARTS tool includes “Technologist A” 22 at an outpatient center A, “Technologist B” 24 at another remote outpatient center B, and “Technologist C” 26 at a hospital emergency room. Each of these remote outpatient centers and/or hospital emergency rooms may also include a front desk clerk/receptionist workstation 28. A PPL operator 30 has access to the ARTS system as well as a reading room assistant 32. Finally, a number of radiologists 34, 36 have access to the ARTS system as will be described in further detail below. It is within the scope of the invention that various sub-roles are also available, such as radiology staff, resident, and fellow.

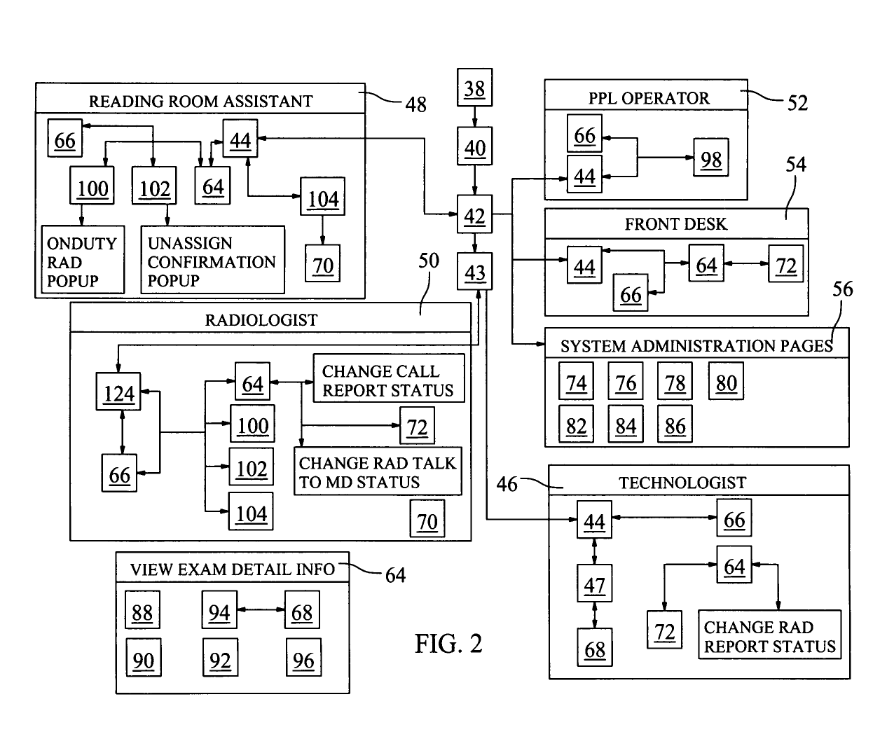

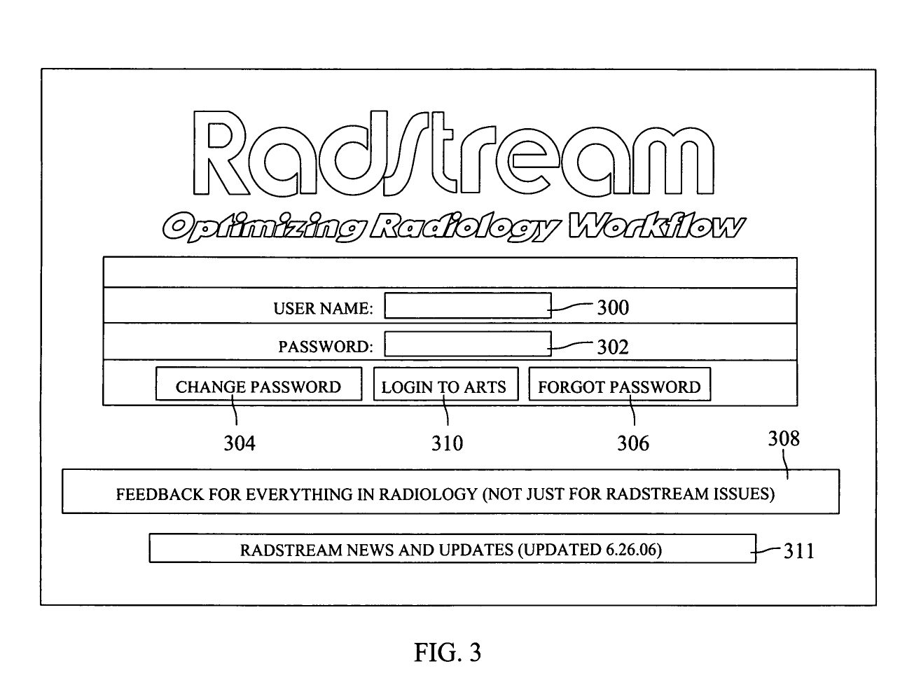

As shown in FIG. 2, operation of the ARTS tool on the server 20initially provides to anyone accessing its home page 38, a login page 40 which is shown in detail in FIG. 3. Once logged in, a roll selection object 42 will be implemented which will either automatically determine the roll of the individual logging in or allow the individual to select a roll from a list of rolls. Users selecting technologist or radiologist roles are directed to a work location and service selection object 43. The user will then be provided with a home page 44 which is personalized for the individual user and the user's roll. In the exemplary embodiment, the available roles include a technologist, a reading room assistant, a radiologist, a PPL operator, a front desk clerk/receptionist, and system administrator.

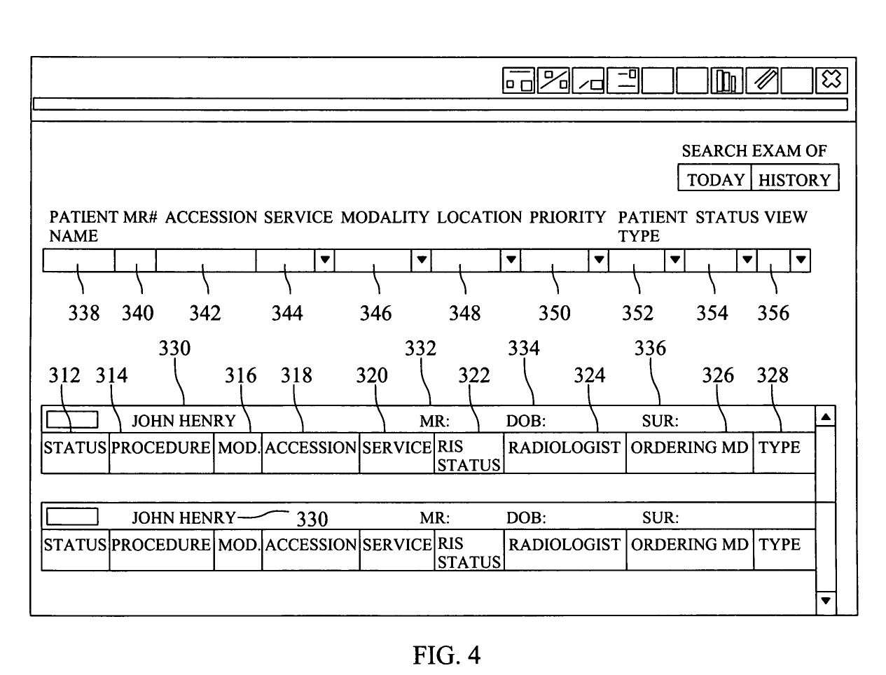

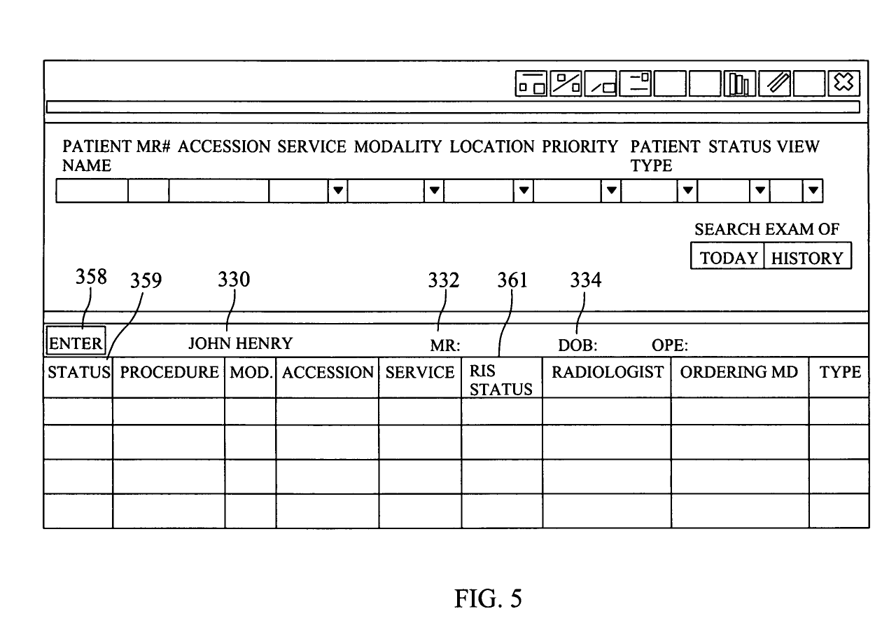

Generally, the process overview includes a technologist (in an outpatient center, for example) accessing a technologist's object 46 on the ARTS system. The technologist views the technologist worklist, shown in detail in FIGS. 4 and 5, to determine the order in which patients should be seen. As disclosed in the abovementioned co-pending application, the worklist displays the patients in priority order. In the exemplary embodiment, the technologist is able to filter the list of patients and exams to show only his or her patients who are to be examined on a certain piece of equipment, thus reducing the amount of irrelevant information displayed. In a further embodiment of the invention, the worklists are automatically generated based on an individual's qualifications and assignments.

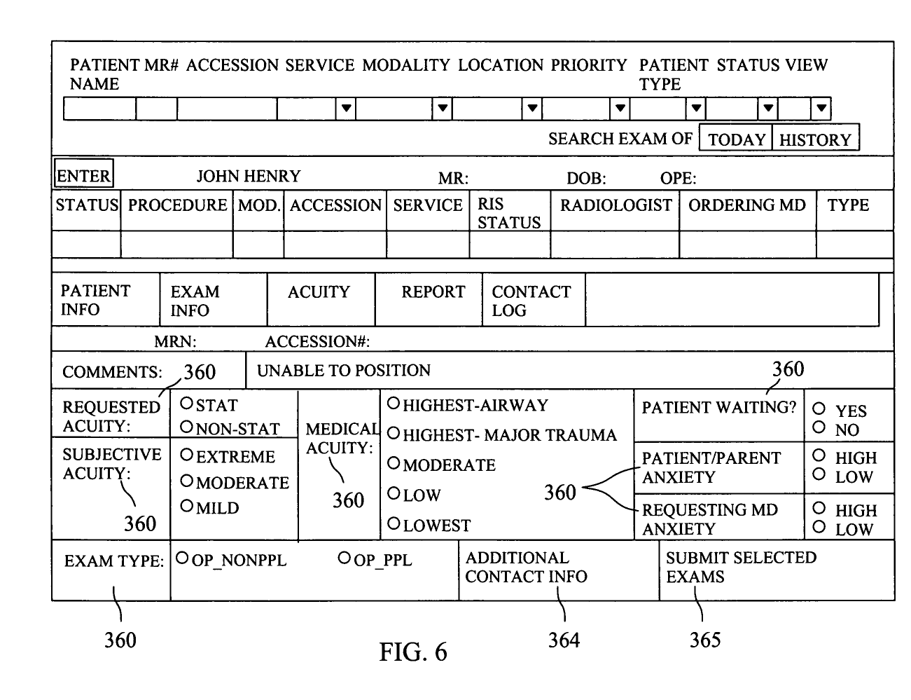

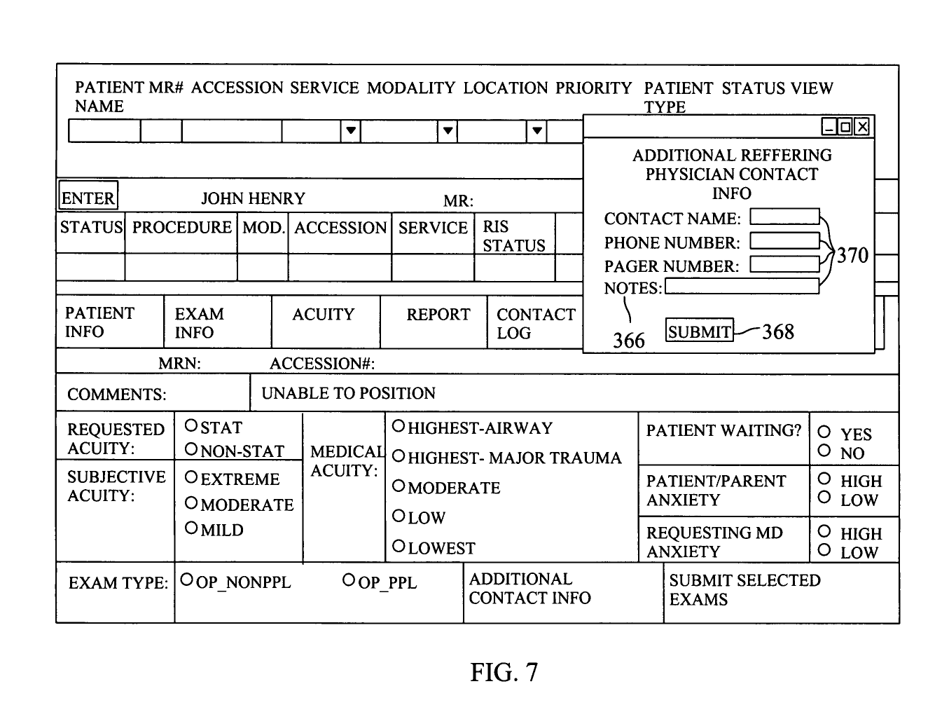

The technologist completes an initial exam of the patient and enters the information using an enter exams object 47. The enter exams object 47 includes inputting certain acuity level factors and optional comments as shown in detail in FIG. 6. The technologist may optionally utilize an additional MD contact info popup 68, shown in detail in FIG. 7, to have the report of the examination sent to another physician. This is useful when the data obtained RIS indicates that only one physician should receive the report of the examination but the patient or the referring physician informs the technologist that the exam report should also be sent to one or more other physicians.

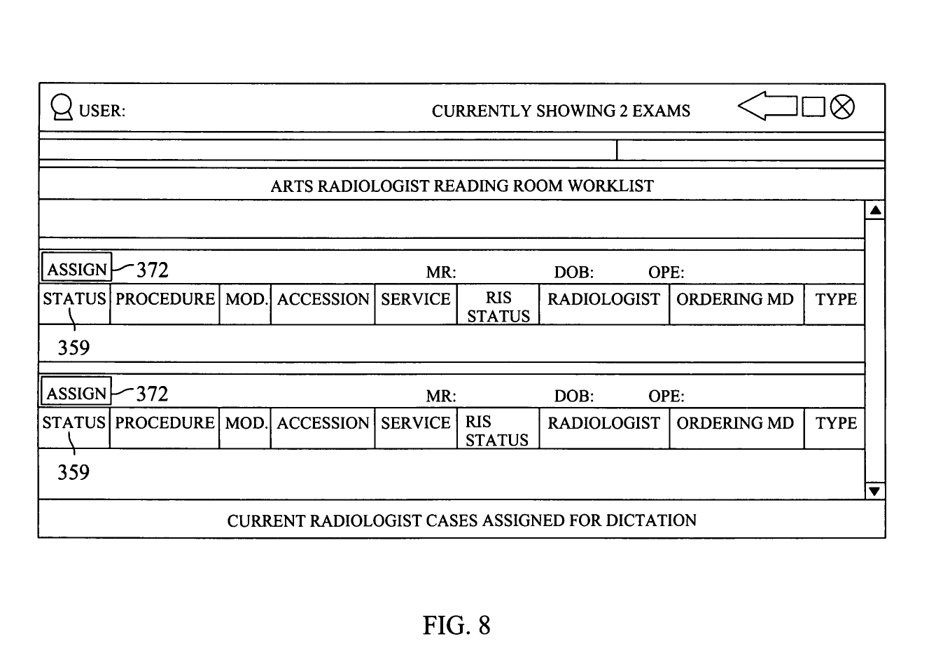

In one exemplary embodiment, a reading room assistant at optional workstation 32 accessing an optional reading room assistant object 48 would monitor the prioritized list of patients, shown in detail in FIG. 8, and assign cases in the prioritized list to available radiologists based upon the priority of the case and the availability of the particular radiologist. Similar to the technologist's worklist, the display can be adjusted to show only the pending examinations of certain types. The reading room assistant utilizes an assign exams object 100 to assign exams and an unassign exams object 102 to unassign exams when necessary. For radiology departments that do not use reading room assistants, the reading room assistant role can be combined with the technologist role. The ARTS system also allows the radiologists at workstations 34 and 36 through the “Radiologist” object 50 to view the prioritized list of patients and to assign cases in the prioritized list to themselves. It is also within the scope of the invention that a radiologist may be assigned to a particular case at any point in the process by any authorized user, including by the technologist or the radiologist himself. This increases overall efficiency because it allows examinations to be directed to a radiologist who is already familiar with a particular patient.

If a reading room assistant is used, he or she may also have available a convey reported exams object 104. The reading room assistant uses this function when he or she speaks with an ordering physician and conveys an examination report. The reading room assistant performs this function for in-house referring physicians.

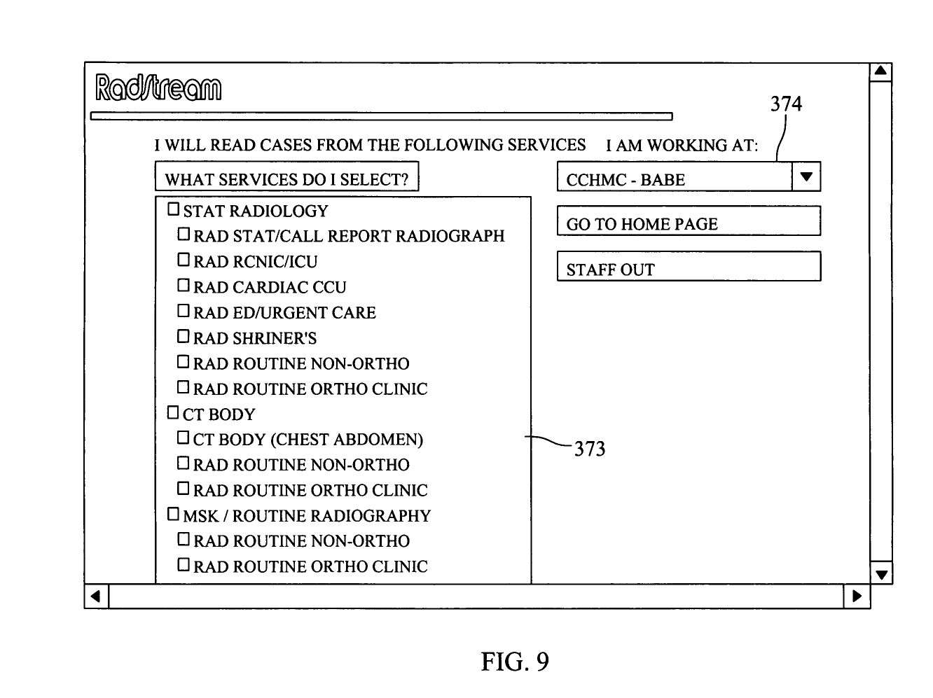

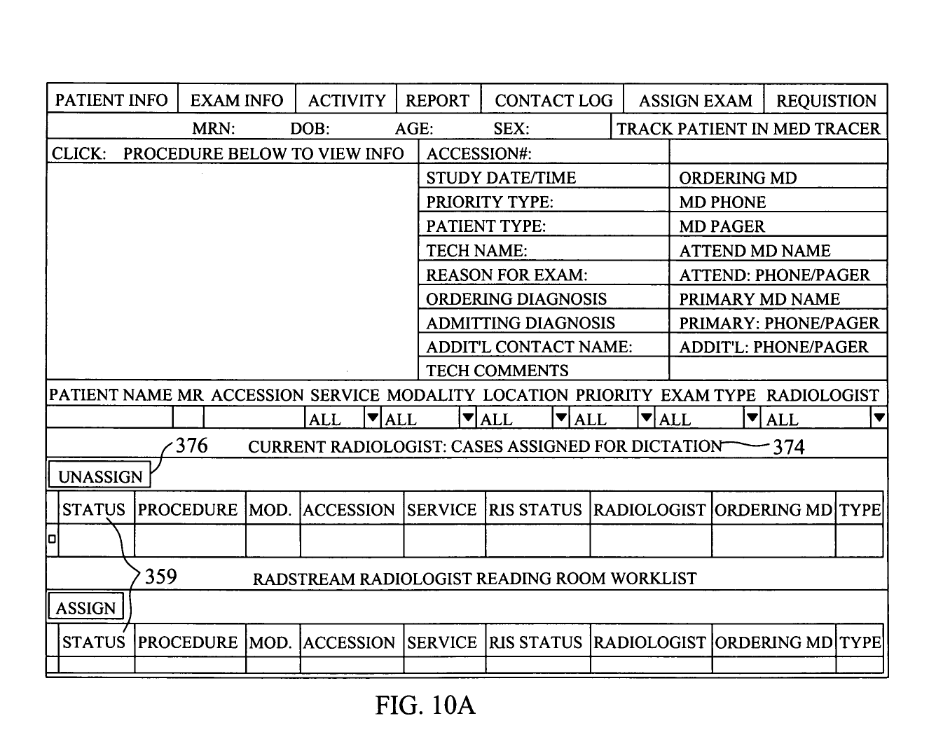

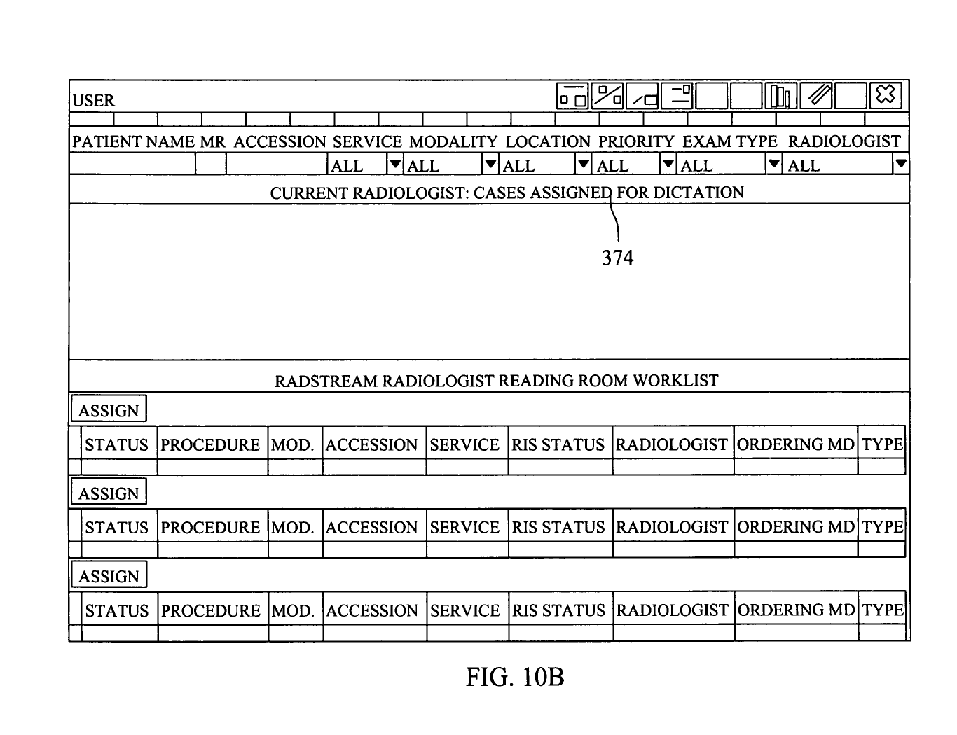

Each radiologist, at workstations 34 and 36, will then access the ARTS system through a radiologist object 50 by selecting his or her physical working location from drop-down box 374 and the types of services he or she will be reading from a list of services 373, both of which are shown in FIG. 9. From here, the radiologist is able to access the assigned list of cases he or she is to examine, shown in FIG. 10a, access all the records and files necessary to perform the particular examination, including any comments from the technologist, and then record his or her report of the examination into the ARTS system, subsequent to which the patient will be removed from the prioritization list and, as appropriate, added to the PPL worklist. As mentioned above, the radiologist can self-assign cases using an assign exams object 100. This capability allows a radiologist to utilize a slow period in his or her workflow to assist a colleague who is receiving a surge of cases, thereby increasing the efficiency of the radiology department as a whole. The radiologist can also unassign an examination by using an unassign exams object 102. A convey reported exams object 104 is available to the radiologist so that he or she can log that the report of an examination has been conveyed to an ordering physician. This function is useful in cases where the radiologist and the ordering physician have a conversation and it is unnecessary for either the reading room assistant or PPL operator to contact the ordering physician to convey the examination report. An add MD contact record 70 may be available for both a reading room assistant and/or a radiologist.

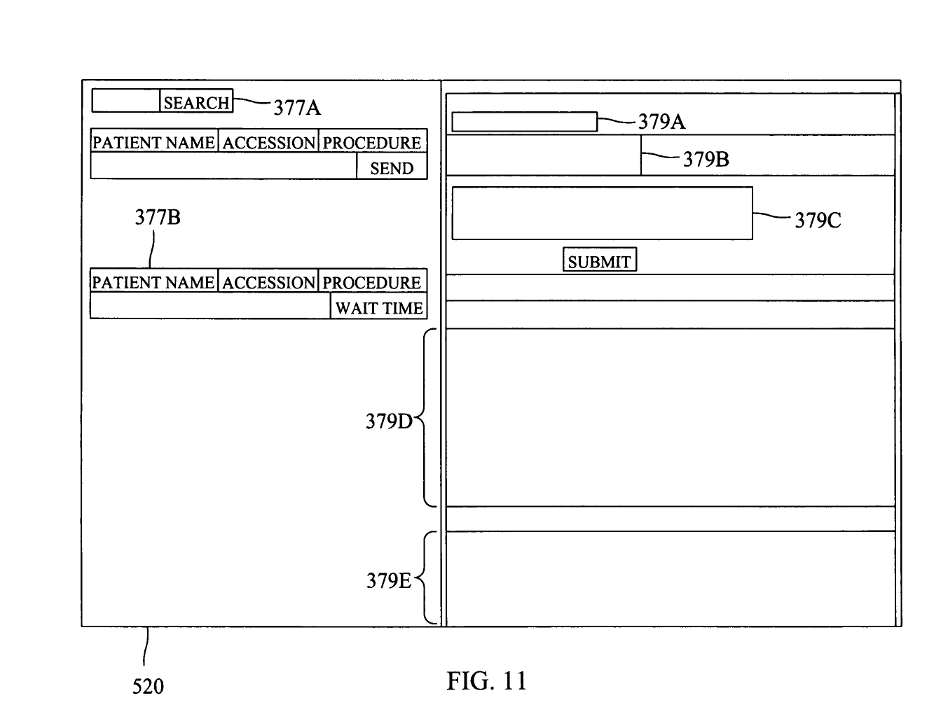

For cases in which more than one practitioner reviews a single exam, such as a supervising radiologist reviewing the report of a trainee or a case in which consultation among two or more radiologists is sought, the present invention provides a communication tool shown in FIG. 11. This tool permits the consulting or reviewing radiologist to send another practitioner a message comparing a preliminary report 379e to a more recent report 379d, along with one or more comments 379c.

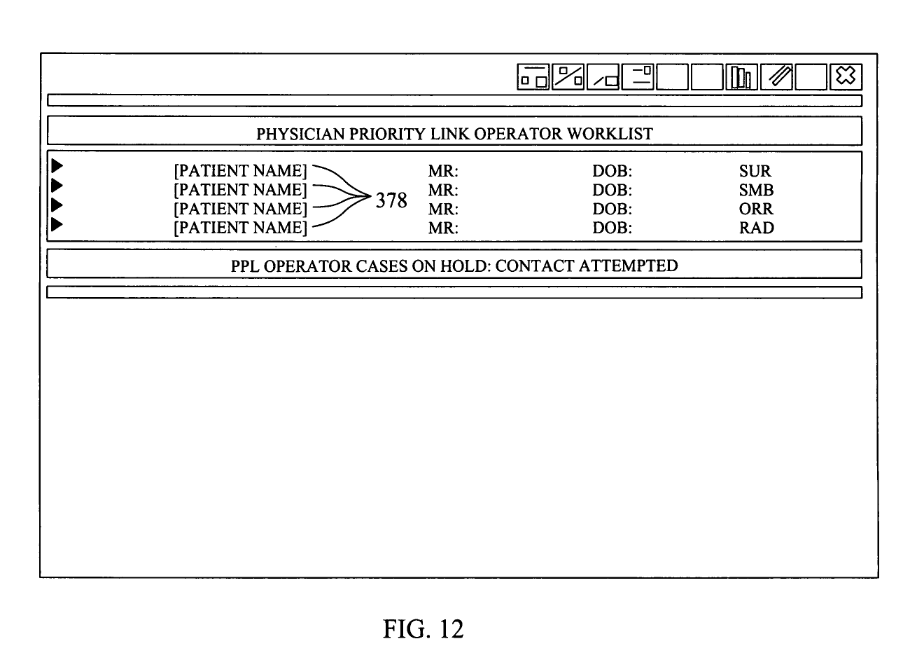

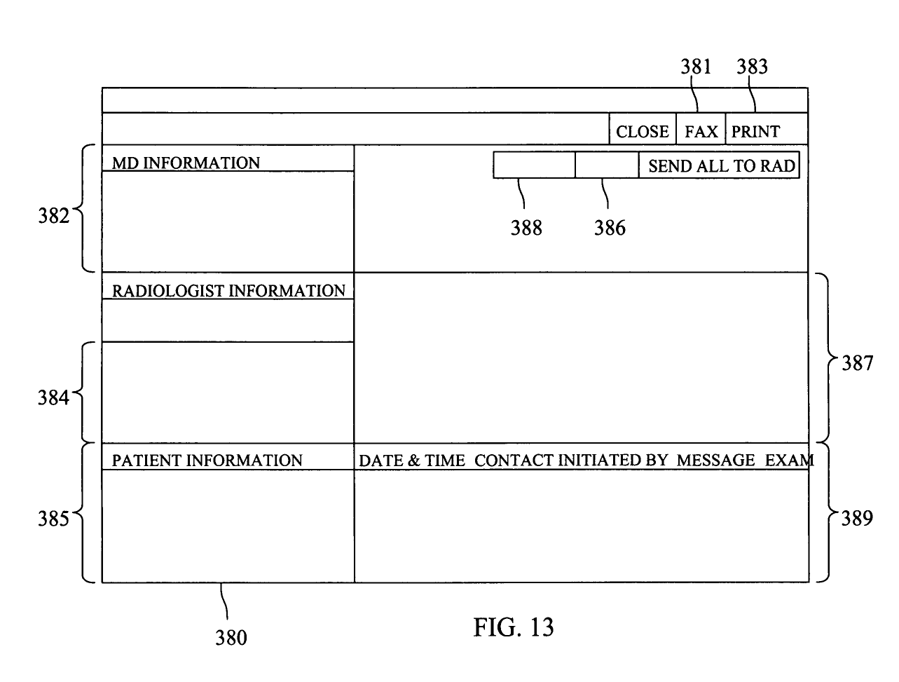

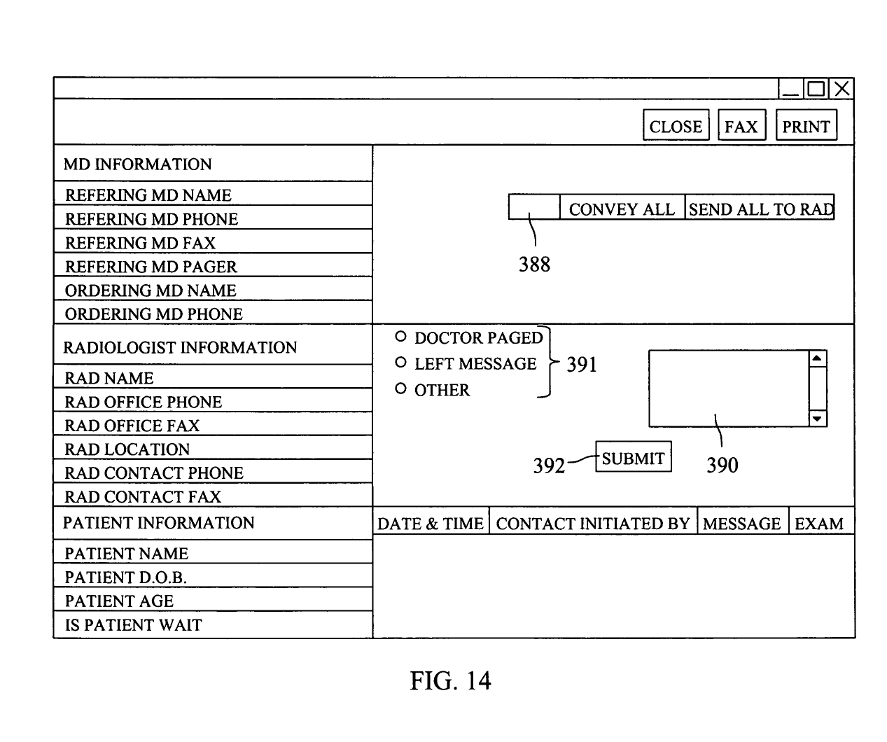

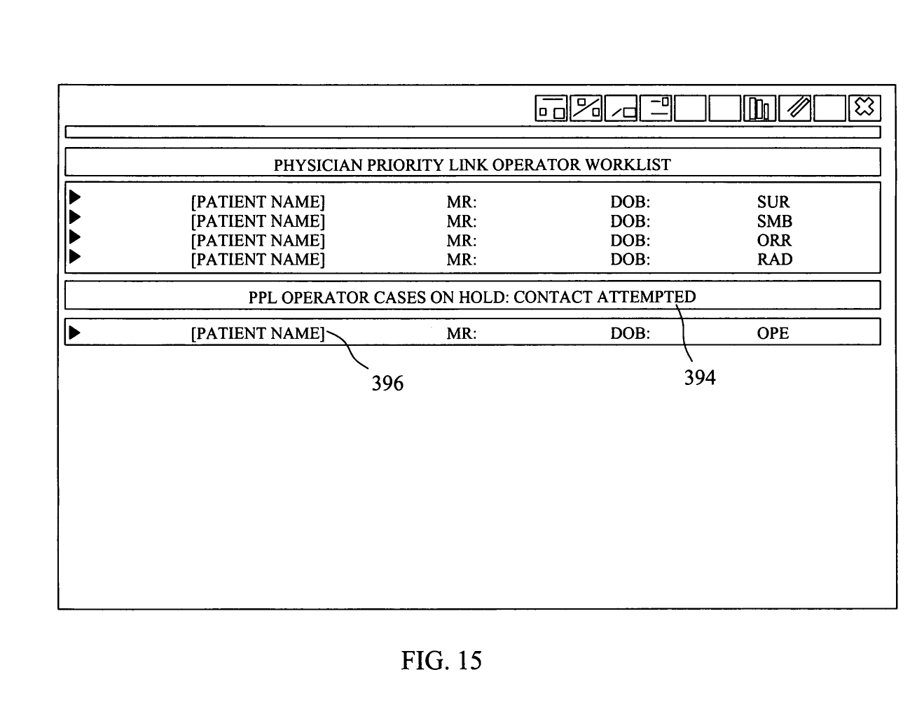

After the radiologist interprets the study and enters the report, either by manually typing it or by using the optional voice recognition capability (or entering it through any other electronic media), the case is transferred off of the radiologist worklist as shown in FIG. 10b to the PPL worklist shown in detail in FIG. 12. The PPL operator views the case information using an exam info popup object 98, shown in detail in FIG. 13, within the “PPL Operator” object 52 on the “PPL Operator” workstation 30. The PPL operator logs any communication or attempted communication with the referring physician using the screen shown in FIG. 14. The PPL operator attempts to contact the ordering physician and convey the results of the examination. If the physician is not available, the PPL operator uses the system to log the attempted communication and the case is transferred to “hold” status, which is effectively another worklist listing cases for which contact has been attempted but the report has not been conveyed to the ordering physician. The PPL operators use the hold worklist to access patient/exam reports when the ordering physician calls back or to reattempt to contact the ordering physician at a later time. A PPL worklist with a case on hold is shown in FIG. 15. The reading room assistants may perform a similar function for in-house referring physicians, or the radiologist may do this himself or herself.

This workflow process allows a decentralized call center to handle asynchronus communications about multiple cases smoothly (that is, any operator “A” may initiate and log a communication attempt; when a response call comes in, any operator “B” can pick up the communication thread for that case, complete it, and log the conveyance). With ARTS, all PPL communication, including routing the case to the PPL operator, occurs in the background, with no effort required by the radiologist. That is, ARTS automatically collects the report from RIS as soon as it's available, routes it to the PPL opertor with all other required information, the PPL operator contacts the referring physician, conveys the report findings, and logs that communication, without involving the radiologist at any point. This is true for both positive and negative STAT cases. It is estimated the radiologist saves approximately 5 minutes per positive STAT case and 135 seconds per negative STAT case by using ARTS instead of the conventional paper-based system. The PPL operators and reading room assistants (who perform the same function as the calling service, contacting in-house referring MDs with reports, brokered by ARTS) save the radiologist an estimated 3-5 minutes for each positive STAT case by being empowered to communicate report results directly to referring MDs, rather than having to connect referring MDs with the radiologist (i.e. paging and waiting for both the referring MD and radiologist to become available simultaneously).

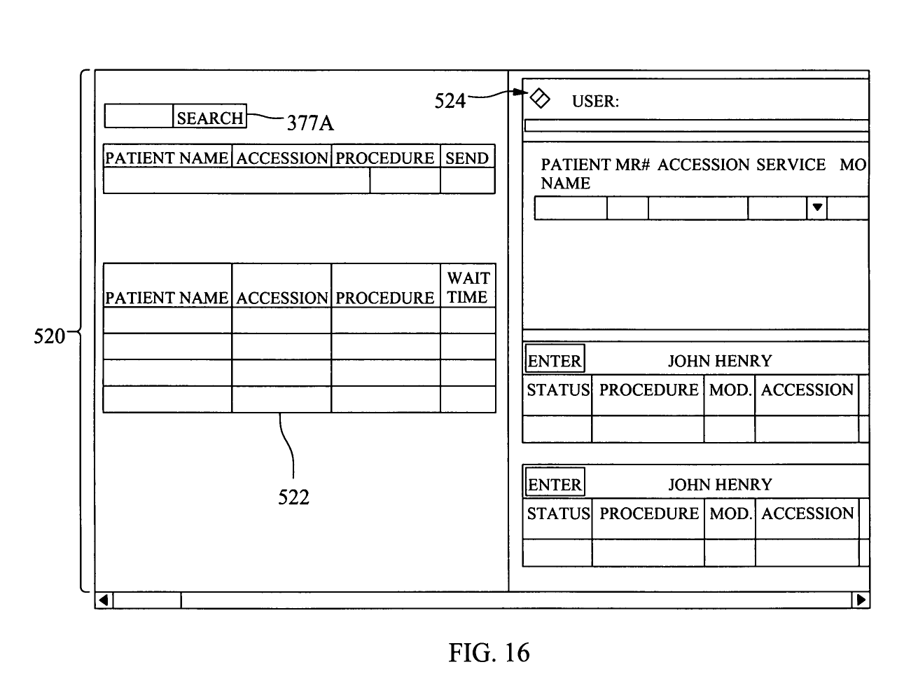

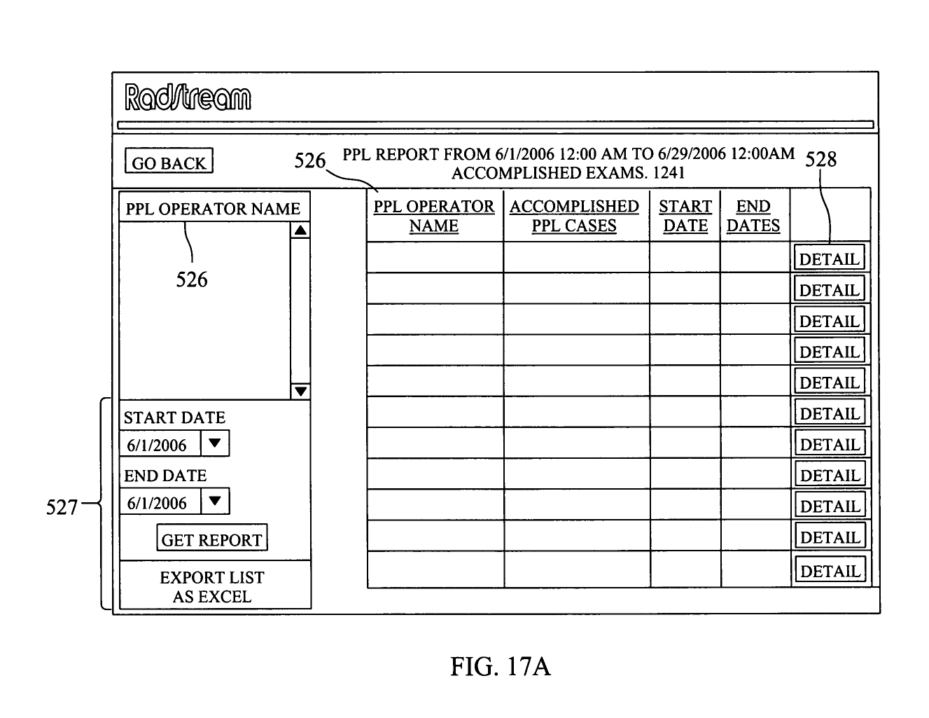

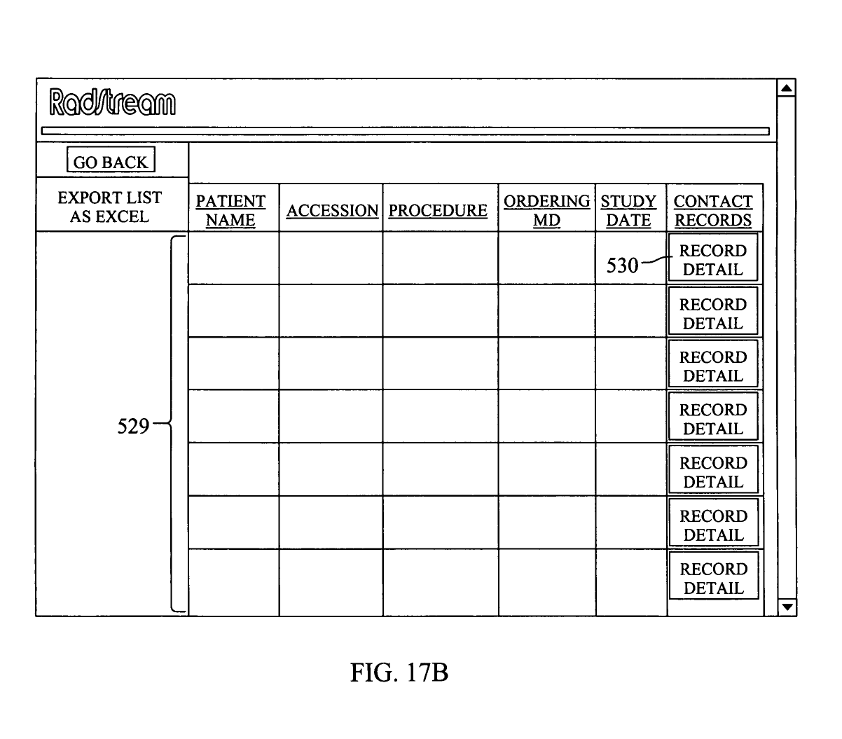

Such conveyance of reports by the PPL operators may be monitored through use of the functions shown in detail in FIGS. 16., 17a and 17b. The radiologists themselves can monitor the status of reported case by opening window 520 shown in detail in FIG. 16. Window 520 provides a status chart 522 that identifies the exams that have been reported, but have not yet been conveyed to the ordering or referring physician. If any one reported exam is not conveyed within 30 minutes of its report, animation 524 spins to alert the radiologist of the delay. Additionally, the conveyance of cases may be monitored according to PPL operator, as shown in detail in FIGS. 17a-17b. By identifying the name of an individual PPL operator 526 and range of dates 527 from the screen shown in FIG. 17a, a user can view the entire list of reports 529 that were conveyed by the PPL operator 526 within the range of dates 527, as shown in FIG. 17b. A detail of each report can be obtained by clicking record “Detail” button 530.

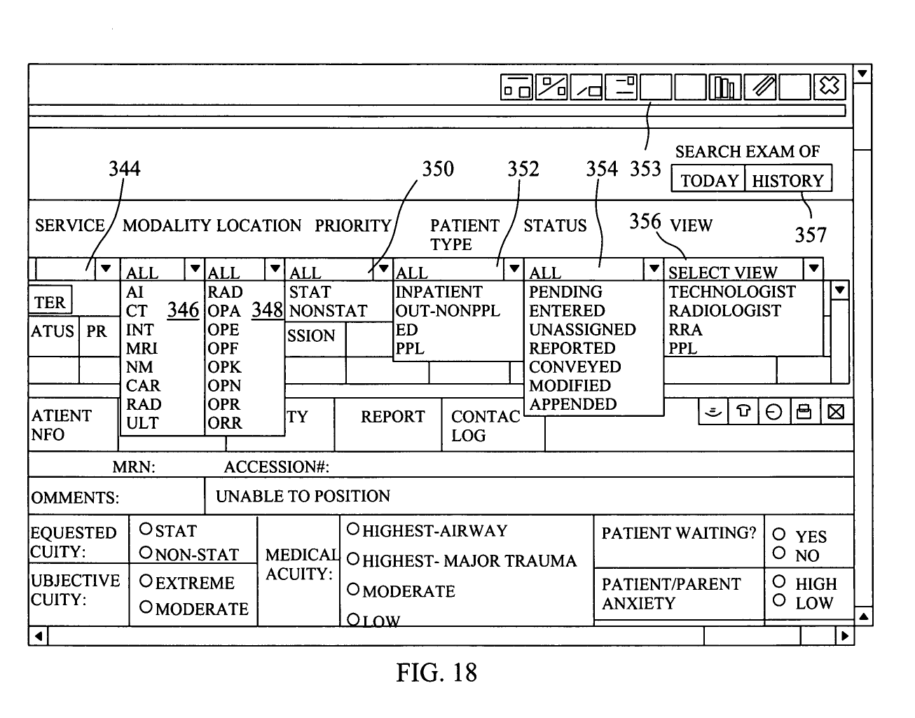

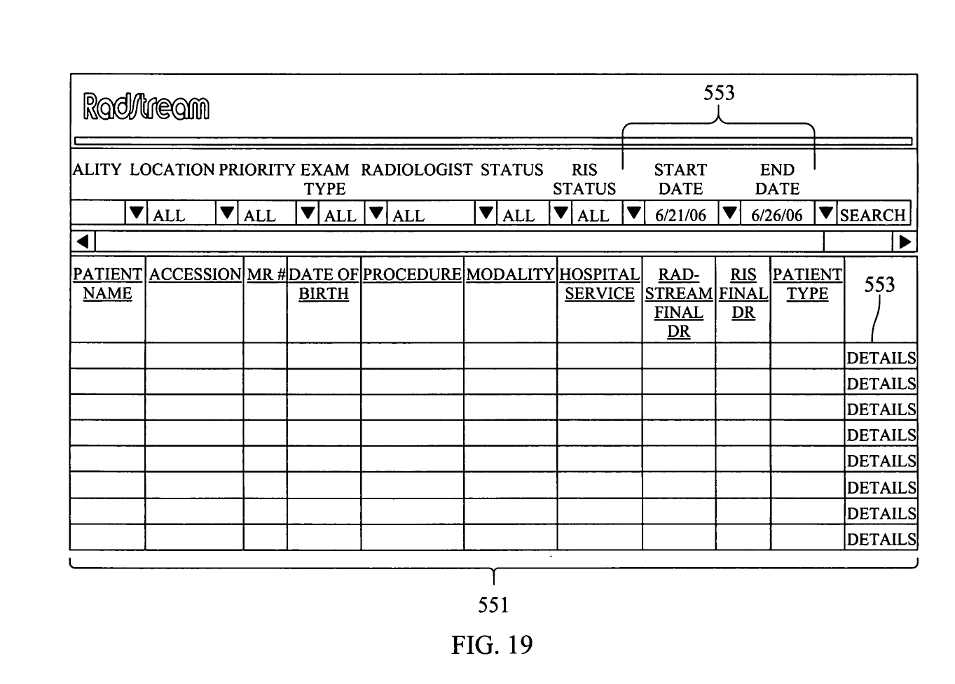

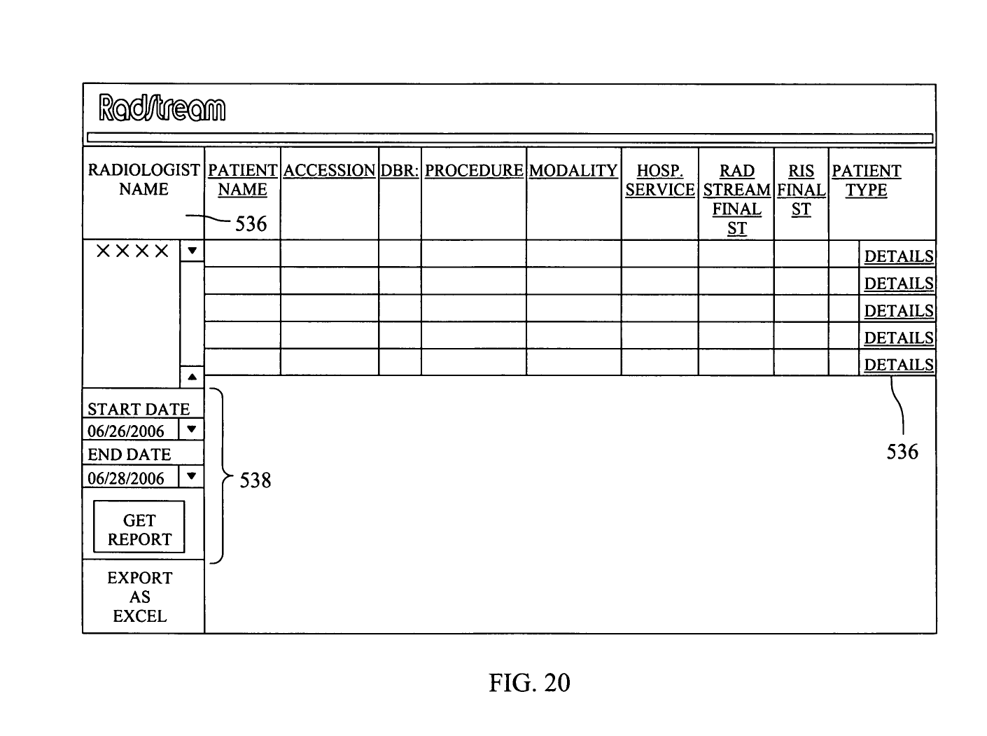

The search all exams object 66 shown under the “Technologist” object in FIG. 2 may be made available to all users. Details of the available search criteria are shown in FIG. 18. Historical records can be searched by clicking on “History” button 357, which take the user to history search screen shown in detail in FIG. 19. A user can easily choose to search only the cases reviewed by a particular radiologist by clicking on the “My Cases” icon 353, which will take the user to the “My Cases” report page shown in FIG. 20. Here, the user can choose the reviewing radiologist name 536 and a range of dates 538.

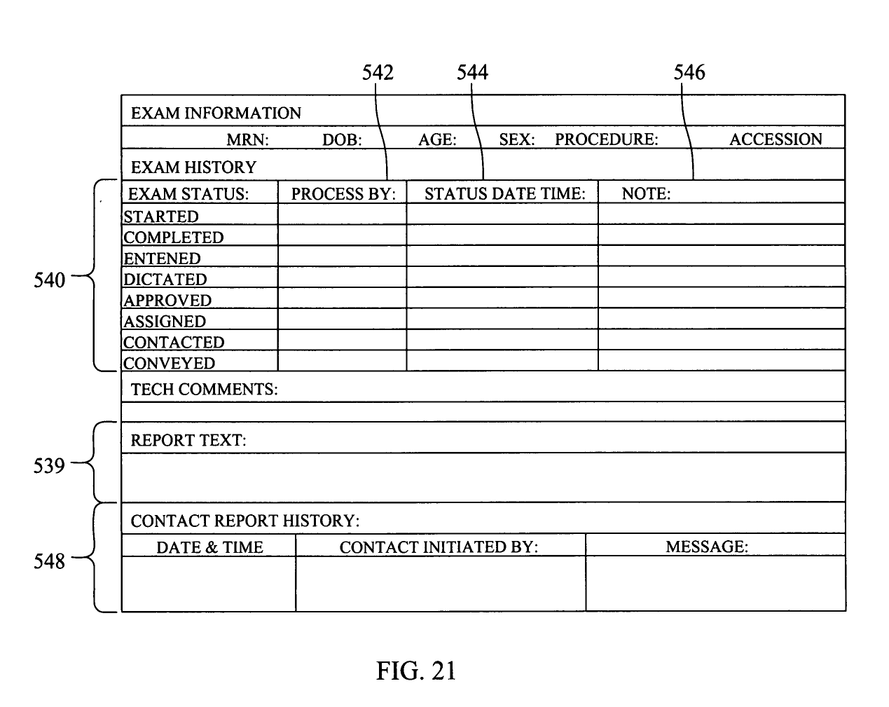

Once a particular examination has been located, the detailed exam information may be viewed by clicking the “Details” button 535 to view all available data about the examination. This takes a user to a screen displaying detailed information relating to an exam at each stage of the process (including “Exam Status” information 540, “Process By” information 542, “Status Date Time” information 544, and “Note” information 546), the “Report Text” 539, and the “Contact Record History” 548. An example of such detail is shown in FIG. 21.

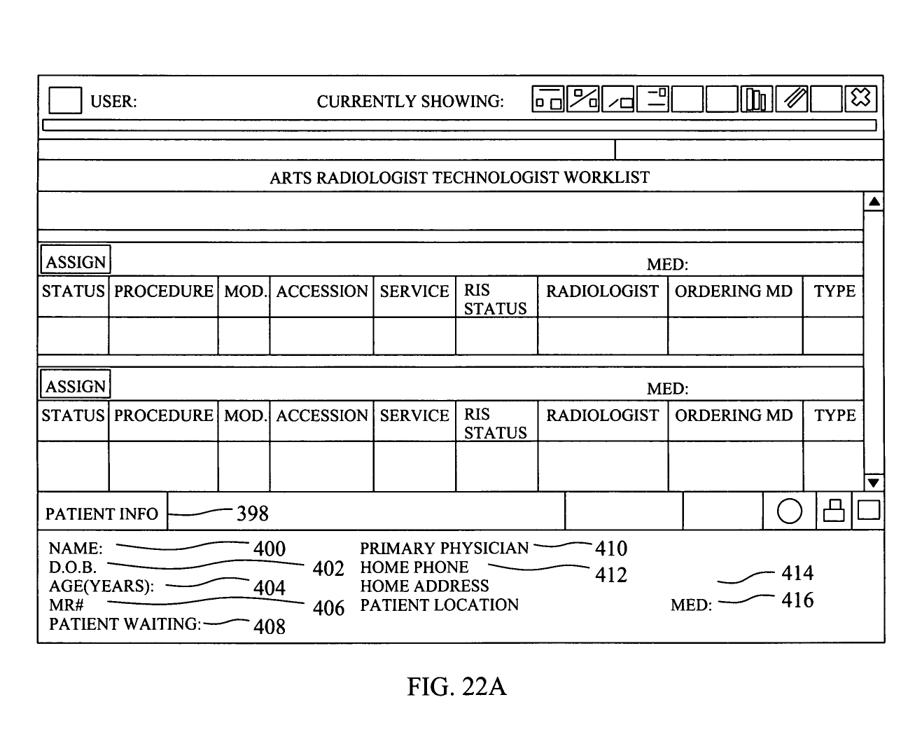

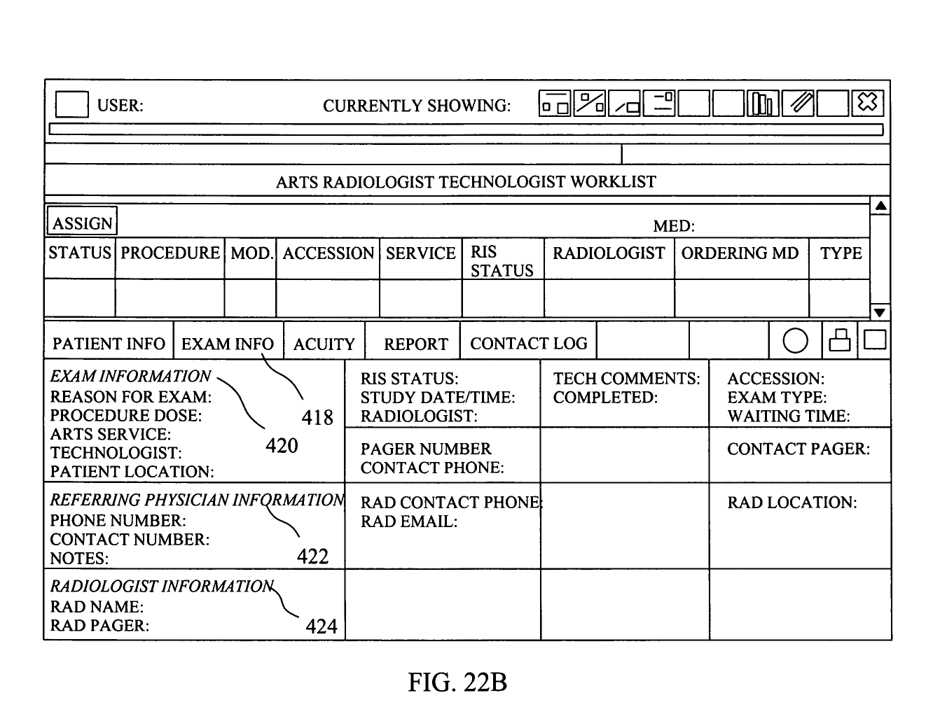

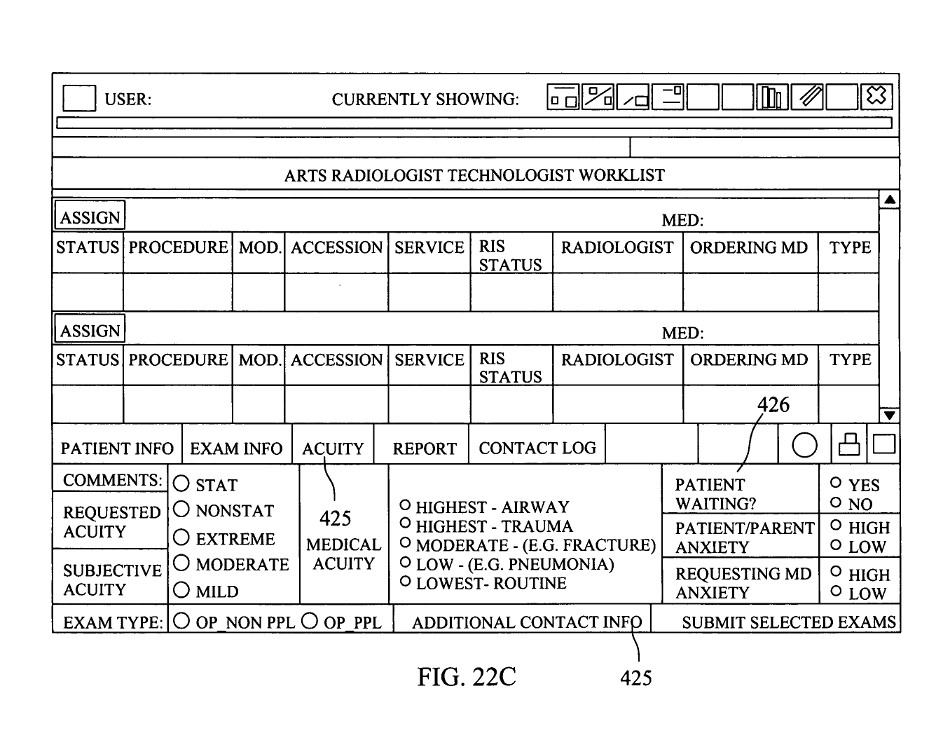

Another way to access information pertaining to a particular exam is to access the “View Exam Detail Info” object 64, which provides patient information 88 (name, date of birth, location, home), examination information 90 (exam, physicians, radiologist, technologist), examination report 92 (patient, physicians, radiologist, exam impression, exam result), exam acuity scores 94 (technologist comment, acuity score, technologist name, service type), additional MD contact information 68, and the exam contact record 96 (message, name and position of person contacted, date, time). Examples of such details are shown in FIGS. 22a-c.

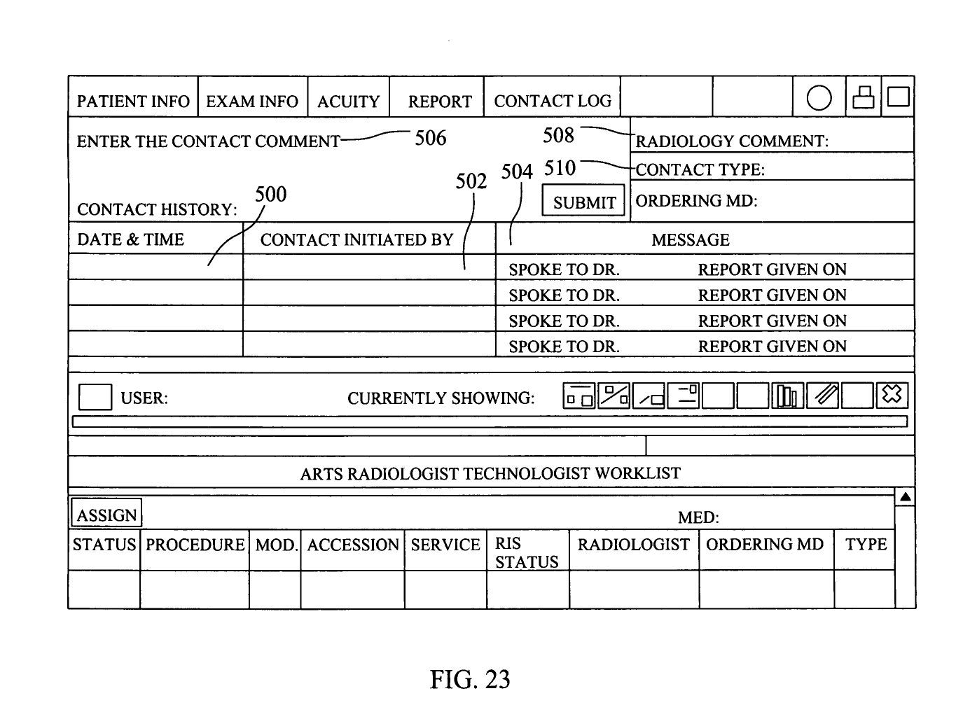

The patient info 88 available for each examination is shown in FIG. 22a. The “Exam Info” 90 available for each exam is shown in FIG. 22b. The “Exam Report” 92 may include all preliminary and final radiologists'reports. FIG. 22c shows the “Acuity” data 94 available for each examination. FIG. 23 shows the “Exam Contact Record” 96 available for each examination.

All users have the ability to change the patient's waiting status using the “Change Patient Waiting Status” object 72. This object provides input into the prioritization algorithm of the abovementioned copending application as well as changing the patient waiting status indication available in the patient's “Exam Acuity Scores” object 94.

The “Add MD Contact Record” 70 may be available in the objects for the “Reading Room Assistant” 48 and “Radiologist” 50. The ARTS system provides the capability for each individual working with the system to provide contact reports such as physician contact records to memorialize all communications between the various individuals for recordkeeping purposes. For example, if a radiologist telephones an ordering physician to discuss a case, the system can be used to record notes of the conversation. This provides a permanent record of the contact that is easily accessible along with other patient records via ARTS.

A similar function is available to other users of the system. For example, if an ordering physician calls to obtain the status of an exam, the system can be used to note the conversation by whichever user speaks to the physician on the telephone. Finally, the Physician Priority Link (“PPL”) operators log each communication and each attempted communication with physicians. All records of communications are automatically time-stamped when they are submitted. The system permanently memorializes both preliminary and final reports examination reports in addition to the communications logs.

The “System Administration Pages” object 56 includes finctions such as “User Management” 74, “Service Management” 76, “Facility Management” 78, contact info management 80, “PPL Report” object 82, “Radiologist Report” object 84, and “Service Report” object 86.

Because all events are logged with the date/time-stamp and user, the system can be used to identify and locate any individual involved in any step of the process. For example a technologist who did not properly complete a study can easily be identified and his or her contact information is available directly from the system. This capability can save a significant amount of time for radiologists and reading room assistants. The date/time-stamping feature also permits operational analysis of the flow of work through the radiology department as well as real-time monitoring of workflow.

FIG. 3 shows an exemplary login screen on which the user is required to input both a user name 300 and a password 302 and then select “Login to ARTS” 310. Additionally, the login screen provides access to functions such as “Change Password” 304, forgotten password retrieval 306, providing feedback to the system administrator 308, and news and updates 311 regarding the invention.

FIG. 4 is an exemplary embodiment of the radiology technologist worklist on a technologist workstation. The patients to be seen by the technologist are listed in priority order as determined by the ARTS prioritization system. For each patient, the worklist displays the patient's name 330, medical record number 332, date of birth 334, and secondary service 336. For each exam, the worklist displays the RadStream “Status” 312, “Procedure” 314, modality 316, “Accession” number 318, “Service” 320, “RIS Status” 322 , “Radiologist” 324, “Ordering MD” 326, and “Type” 328. Some fields are blank because the applicable data is obtained at a later step in the process (e.g., the radiologist field 324 is filled when a radiologist is assigned to interpret the exam). Additionally, the technologist can search all exams. Fields of search include “Patient Name” 338, medical record number 340, “Accession” number 342, “Service” 344, “Modality” 346, “Location” 348, “Priority” 350, “Patient Type” 352, “Status” 354, and “View” 356. It is within the scope of the invention that the search fields may include drop-down menus listing available subheadings, as shown in FIG. 18.

FIG. 5 shows an exemplary screen shot of the technologist workstation. The status column 359 indicates “Reported” for a previously dictated examination and “Pending” for the examinations that are ready for the technologist to enter in ARTS. The “RIS Status” column 361 indicates “Approved” for the previously dictated and signed examination and “Completed” for the exam just completed in RIS by the technologist. The technologist selects “Enter” 358 to populate the acuity score screen.

FIG. 6 shows an exemplary screen shot of the acuity data viewed on a technologist workstation. The technologist can view or update the answers to questions 360 which will be used by ARTS to determine the patient's position in the priority list. The technologist also specifies whether the examination report is to be conveyed to the ordering physician via the PPL. Additionally, the technologist may optionally review or enter comments in field 362. For example, a technologist may add a comment stating that a patient was not able to be properly positioned during an examination due to pain. In the past, this would be communicated to the radiologist via a written note or a telephone call. The present invention allows the technologist to type the comment directly into the computer system. The radiologist views the technologist's comments when he or she selects the case for interpretation. The present invention increases efficiency because the comment accompanies all of the other patient information and examination data sent to the radiologist. The radiologist is not disturbed by a phone call and the note cannot be misplaced. All comments are automatically time-stamped when they are submitted. It is within the scope of the invention that comments and other information may be input directly into the system using voice recognition software (or other form of electronic media), reducing the time that personnel spend on administrative tasks.

The technologist may also select “Additional Contact Info” 364 to have the report of the exam sent to an additional physician (see FIG. 7). When all of the data entry and review is complete, the technologist selects “Submit Selected Exams” 365 to send the completed examinations to the radiologist's worklist.

FIG. 7 shows an exemplary screen shot of the “Additional Contact Info” function 366. The technologist enters information in fields 370 for the physician who should receive the report and selects “Submit” 368.

FIG. 8 shows an exemplary screen shot of the radiologist reading room worklist. This list of unassigned cases for examination by a radiologist may be accessible by both the reading room assistant and the radiologists. The exams are listed in order of acuity as determined by ARTS. The “Status” column 359 indicates “Entered,” meaning that the examinations are ready for interpretation by a radiologist. The “Assign” buttons 372 allow the reading room assistant, technologist, or radiologists to assign cases to specific radiologists for interpretation and dictation. In addition to viewing the list of cases ready for interpretation, the reading room assistant can monitor for examinations that have been entered into ARTS but have not been completed in RIS. This capability prevents an examination from being inadvertently overlooked for a long period of time due to a minor error when the examination is obtained. When the reading room assistant is alerted to a case in this status, he or she contacts the technologist who corrects the problem, and the case is removed from the alert list.

FIG. 9 shows an exemplary screen shot of the screen that may be used by individual radiologists to identify the physical location at which he or she will review exams, and choose the type of exams he or she will review from a list of exams 373. It is within the scope of the invention for physical location to be identified from a drop-down box 374 containing a list of locations for a particular health system or network of health systems.

FIGS. 10a shows other exemplary screen shots of the reading room worklist. Under the heading “Current Radiologist: Cases Assigned for Dictation” 374 are listed the cases assigned to the radiologist who is logged in to the system. The radiologist can remove a case from his or her worklist by selecting the “UnAssign ” button 376, which will move the case back to the list of examinations awaiting assignment to a radiologist and change its status to “Entered.” The “Status” column 359 under the “Cases Assigned for Dictation” 374 indicates “Assigned,” meaning that the case has been assigned to a radiologist for interpretation. All ARTS screens then indicate that the assigned radiologist is actively interpreting the study. This function prevents wasted time due to more than one radiologist commencing an interpretation of a case and later discovering that his or her colleague has also begun an interpretation of the same case. Once the interpretation is complete, the radiologist either manually types the report into the system or dictates it using optional voice recognition software.

FIG. 10b shows an exemplary screen shot of the reading room worklist after the case assigned to the radiologist was interpreted and dictated. The case was removed from the “Current Radiologist: Cases Assigned for Dictation” 374 list and was transferred to the PPL worklist shown on FIG. 12.

FIG. 11 shows an exemplary screen shot of a communication tool feature that may be used when more than one radiologist reviews a single exam. Using this tool, a consulting or supervising radiologist may search for particular exam for consult or education by using search box 377a. When the target exam is found and reviewed by the consulting or supervising radiologist, he or she can send a comparison of a “Preliminary Report” 379e and a more recent report 376d to a second radiologist. It is within the scope of the invention that the second radiologist may be identified from a drop-down box 379a listing radiologists of a particular health system or network of health systems. The radiologist sending the comparison may also choose a reason for the comparison 379b and/or enter comments regarding the comparison in comment field 379c. It is also within the scope of the invention that the consulting or supervising radiologist can enter any type of comment in the commend field 379c based merely upon an evaluation of the target exam (with or without a comparison to another exam), or based upon other considerations.

FIG. 12 shows an exemplary embodiment of the PPL operator worklist on the PPL operator workstation. An examination report that is to be conveyed to the ordering physician via the PPL (as specified by the technologist on the screen shown in FIG. 6) automatically appears on the PPL worklist when it is ready to be conveyed. ARTS automatically obtains the examination report from RIS and makes it available to the PPL operator. The PPL operator views the details of a case by selecting from a list of patients'names 378. It is within the scope of the invention that the PPL operators are signaled that a new case has appeared on their worklist by an audible alert.

FIG. 13 shows an exemplary screen shot of the “PPL worklist detail” 380 of the case selected by the PPL operator. The PPL operator has access to contact information for the ordering and referring physicians 382 and the radiologist 384 who interpreted the exam, as well as patient information 385. The PPL operator will attempt to contact the ordering physician. If the physician is available and the report 387 is conveyed, the PPL operator selects “Convey all” 386, causing the case to be removed from the worklist and populating the contact log. If the physician is not available to receive the report, the PPL operator selects “Hold all” 388. If there is a previous contact record it appears on the contact history 389 portion of the screen. The system also allows the PPL operator to fax or print the report by selecting the appropriate button, 381 or 383.

FIG. 14 shows an exemplary screen shot of the PPL operator worklist detail screen which is accessed when the PPL operator selects “Hold all” 388. The PPL operator selects one of four options 391, types a brief note about the attempted contact 390, and then selects “Submit” 392. The case is moved to the “PPL Operator Cases on Hold: Contact Attempted” list 394 as shown in FIG. 15. If the physician calls the PPL facility to obtain the examination report, any PPL operator can access the case detail screen by selecting the case 396 from the list, and see all prior contact attempt information as well as all exam information. That PPL operator then “conveys” the examination which then disappears from the active PPL worklist screen. The reading room assistant performs similar functions for in-house referring physicians.

FIG. 16 shows an exemplary screen shot of panel 520 hat may be used by a radiologist to monitor the status of exams that have been reported by the radiologist, but have yet to be conveyed by the PP1 operator. Panel 520 provides a chart 522 identifying the exams yet to be conveyed and the wait time for each. FIG. 16 also shows animation 524 which, according to an exemplary embodiment, “spins” to alert the radiologist if any one reported exam has not been conveyed within a specified length of time.

FIGS. 17a-17b show yet another exemplary screen for monitoring the conveyance of reported exams. The exemplary screen shown in FIG. 17a permits a user to search conveyance records by PPL operator 526, within a specified range of dates 527. To choose the records for a specific PPL operator, the user may click “Detail” button 528, which would take the user to a screen such as that shown in FIG. 17b. From here, the user may choose from a list of exams 529 conveyed by the chosen PPL operator by clicking on “Record Detail” button 530.

FIG. 18 shows an exemplary screen shot of the search functions available to any user. Searches can be performed by “Patient Name” 338, medical record number 340, “Accession” number 342, “Service” 344, “Modality” 346, “Location” 348, “Priority” 350, “Patient Type” 352, “Status” 354, and “View” 356. The search function allows users to search and review entered, pending, reported, completed, and conveyed exams. Drop down menus may provide available subheadings. Alternatively, a user may search historical exam records by clicking “History” button 357, which would take the user to a screen such as that shown in FIG. 19. From here, the user may access a list of exams 351 pertaining to a specified range of dates 532. To view the detail information for any of the exams, the user may click the “Details” button 535 corresponding to the chosen exam. Yet another way in a user may search the exams records is by activating the “My Cases” icon 353 (see FIG. 18). This would take the user to a screen such as that shown in FIG. 20, from which the user may choose from a list of reviewing radiologists 536 and specify a range of dates 538. Again, to view the details from any of the resulting list of exam records, the user need only click on the corresponding “Details” button 535. An example of such detail is shown in FIG. 21, and provides, in addition to the text of the exam report 539, the status history 540 of the exam as it was processed, the person 542 who performed each step in the processing of the exam, the date and time 544 of each stage in the process, and any notes 546 corresponding to each stage in the processing. Any messages that were sent through the system regarding the exam are also noted under the “Contact Record History” heading 548.

Because the search function is available to any user, any user can respond to a request for the status of an examination. This increases efficiency of the department as a whole because the radiologists are not disturbed by calls requesting status. Customers are more satisfied because they can learn the status of their exams quickly without having their phone calls transferred numerous times.

In another example of the application of this capability, the front desk clerk/receptionist can search for the real-time status of any examination at the request of a patient. The front desk clerk/receptionist can use the data retrieved to inform the patient of the current status of his or her radiology report(s), help the patient decide whether to wait, and inform him or her when to expect a call from his or her physician, whether radiology has begun to try to communicate the examination results to his or her physician, and whether the physician was successfully contacted.

FIG. 22a shows an exemplary screen shot of the RIS “Patient Info” 398 accessible through ARTS. All RIS patient data is available for viewing using ARTS. The data includes the patient's name 400, date of birth 402, age 404, medical record number 406, waiting status 408, primary physician 410, home phone number 412, home address 414, and current location 416.

FIG. 22b shows an exemplary screen shot of “Exam Info” 418 accessible through ARTS. All examination data available from RIS can be viewed using ARTS. The data includes specific information about each exam 420, as well as physician information 422 and radiologist information 424. It is also within the scope of the invention that contact information is available in the system for various personnel involved in the gathering, interpretation, and reporting of radiology examinations, such as technologists, radiologists, and referring physicians.

FIG. 22c shows an exemplary screen shot of a patient's “Acuity” data page 425. The “Patient Waiting” status 426 can be updated at any time by any user, including the front desk clerk. It is within the scope of the invention that the estimated time to radiology report availability as calculated by the system disclosed in the abovementioned copending application is available to all users.

An exemplary contact log is shown in FIG. 23 including the date/time stamp 500, user 502, and message 504 relating to each contact made regarding a particular exam. The screen allows another contact record to be entered using the comment block 506, radiology contact 508, and contact time 510.

Summary of advantages of ARTS communications and workflow functions:

| Function | Feature Note | Potential Impact |
|---|---|---|
| Paperless workflow | Eliminates paper | Smooth Workflow |
| | requisitions | |
| | | No lost requisitions |
| | | Legible technologist notes |
| | | Decreased confusion |
| | | Decentralized study acquisition |
| | | Decentralized study interpretation |
| | | Real-time workload balancing |
| Transparent workflow | Workflow surges addressed | If one area is swamped, less busy |
| | early | radiologists can step in |
| | | Proactive elimination of bottlenecks |
| | | Improved patient care |
| Automated triage | Ensures appropriate and | Patients treated first: |
| | timely delivery of care | sickest |
| | | needing subsequent tests |
| | | needing further treatment |
| | | waiting for discharge |
| | | Improved patient care |
| | | Improved hospital workflow |
| | | Improved patient satisfaction |
| | | Increased efficiency of care |
| | | Increased referrals |
| | | Increased revenue |
| Effective electronic | Supplies pager number, | Reduced radiologist and referring |
| tools to contact | phone numbers, and brokers | physician frustration |
| referring physicians | contact between radiologists | |
| | and referring physicians | |
| | To communicate important | Improved communication |
| | findings or questions | |
| | | Reduced workflow interruptions |
| | | Increased efficiency |
| | | Improved patient care, satisfaction |
| Communication tools | Covers important | Improved clarity as to which |
| | information in addition to | caregivers have been notified of |
| | RIS status and radiology | critical findings |
| | reports | |
| | Facilitated communication | Simplified process for |
| | | communicating results - reduced |
| | | medical errors |
| | Permanent communication | Permanent log of what was said to |
| | log | whom, and when |
| | Also logs unanswered | Improved medicolegal |
| | pages, and calls never | documentation in the event of |
| | returned to radiology | communication failures: by |
| | | documenting failed and repeated |
| | | attempts by radiology to reach |
| | | responsible caregivers, system |
| | | offers increased medicolegal |
| | | protection to radiology |
| | | Reduced duplication of efforts by |
| | | staff to communicate important |
| | | findings |
| Location and status of | Useful to caregivers both | Enhanced tracking of patients |
| patients throughout | within and outside the | throughout radiology workflow |
| department | radiology department | |
| | | Allows any radiology staff member |
| | | to answer calls from referring |
| | | physicians regarding patient |
| | | location and study status - |
| | | eliminates multiple call transfers, |
| | | eliminates time wasted locating |
| | | patients |
| | | Enhanced radiology department |
| | | image as providing coordinated, |
| | | organized, informed care |
| | | Increased referrals |
| Waiting status of | Does patient need final | Improved communication between |
| patient | radiology report or to speak | patients and their physicians |
| | to their physician before | |
| | leaving department? | |
| | | No “orphaned” patients in the |
| | | waiting room |
| | | Shorter wait times |
| | | Improved patient and referring |
| | | physician satisfaction |
| | | Increased referrals |
| Electronic capture of | Examples: “Patient was | Improved communication between |
| technologist comments | difficult to position”, | technologists and radiologists |
| and notes | “Please send copy of report | |
| | to Dr. Smith”, “Pain located | |
| | at base of thumb” | |
| | | Eliminates loss of handwritten notes |
| | | Guarantees that appropriate |
| | | information is available to the |
| | | radiologist at time of interpretation |
| | | Improved patient care |
| Contact Information | Who is included: | Time Saver |
| | Radiologist | |
| | Technologist | |
| | Referring physician | |
| | Patient | |
| | What is included: | Decreased frustration |
| | Pager Numbers | |
| | Phone Numbers | |
| | Locations | |
| | | Users can locate staff quickly |
| | | Users can contact staff quickly |
| | | Clarifies which staff is involved in |
| | | each case |
| | | Improved communication |
| | | Improved patient care |
| Permanent capture of | Useful both to private | Increased clarity regarding changes |
| all preliminary reports, | practices and academic | to radiology reports |
| subsequent versions, | centers | |
| and addenda | | |
| | | Documents who said what, when, |
| | | and to whom |
| | | Eliminates ambiguity about |
| | | radiology resident preliminary |
| | | interpretations |
| | | Clarifies timing of different report |
| | | versions |
| | | Decreases medicolegal exposure |

Following from the above description and invention summaries, it should be apparent to those of ordinary skill in the art that, while the systems and processes herein described constitute exemplary embodiments of the present invention, it is to be understood that the invention is not limited to these precise systems and processes and that changes may be made therein without departing from the scope of the invention as defined by the claims. For example, while the exemplary embodiments are described with reference to a radiology case management system, it will be apparent to those of ordinary skill in the art that other medical (or even non-medical) case management systems (such as, for example and without limitation, emergency room case management systems, pharmacy case management systems, medical testing case management systems, and the like) will also fall within the scope of certain aspects of the present invention as claimed.

Additionally, it is to be understood that the invention is defined by the claims and it is not intended that any limitations or elements describing the exemplary embodiments set forth herein are to be incorporated into the meaning of the claims unless such limitations or elements are explicitly listed in the claims. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A computer assisted method for tracking the progress of patient-related activity in a medical institution, comprising the steps of:
providing a computerized system having access to electronic records that include patient data and requested task data for each of a plurality of patients, the requested task data including at least one of ordered examination data and ordered procedure data;
assigning with the assistance of the computerized system a requested task to a medical institution professional based at least upon a task type for the requested task and at least upon a responsibility assigned to the medical institution professional;
selectively displaying on a graphical user interface associated with the computerized system one or more requested tasks assigned to the medical institution professional along with an identity of the patient associated with the one or more requested tasks;
updating the electronic records with assignment information concerning the assigning step;
upon substantial completion of at least one of a requested task and a step involved in the requested task, logging, by at least one of the medical institution professional and an assistant to the medical institution professional, with the assistance of a graphical user interface provided by the computerized system, completion information concerning the at least one requested task and step involved in the requested task; and
updating the electronic records with completion information concerning the completion step.

2. The method of claim 1, further comprising the steps of:
subsequent to the substantial completion of at least one of the requested task and the step involved in the requested task, accessing result information associated with the requested task with the assistance of a graphical user interface provided by the computerized system;
communicating the result information to a third party; and
updating the electronic records with communication information concerning the communicating step.

3. The method of claim 2, further comprising a step of selectively searching for at least one of assignment information, completion information and communication information utilizing a computerized search engine provided by the computerized system.

4. The method of claim 3, wherein the searching step is based upon search criteria including one or more of the following search criteria:
patient name;
record number;
service;
modality;
location of requested task;
priority;
patient type;
status of requested task;
date of requested task;
healthcare provider associated with the requested task; and
medical institution professional associated with the requested task.

5. The method of claim 2, wherein the assignment information, completion information and communication information include a date and time stamp.

6. The method of claim 2, wherein the communication information includes information concerning identity of an initiating party and identity of the third party.

7. The method of claim 6, wherein the communication information includes communication content information entered by the initiating party using a graphical user interface associated with the computerized system.

8. The method of claim 1, wherein the assignment information and completion information include a date and time stamp.

9. The method of claim 1, wherein the assignment information includes information concerning the identity of the medical institution professional.

10. The method of claim 9, wherein the assignment information includes information concerning identity of a party who initiated the assigning step.

11. The method of claim 1, wherein the completion information includes information concerning identity of a party who initiated the logging step.

12. A computerized method for generating individualized lists of requested tasks assigned to a medical institution professionals in a medical institution, comprising:
comparing, by a computerized system, an electronic list of requested tasks to be completed against an electronic list of medical institution professionals available to perform the requested tasks, the comparing step including a step of considering types of tasks that each of the medical institution professionals is qualified and assigned to perform;
assigning, with at least the assistance of the computerized system a plurality of the requested tasks to at least one medical institution professional based upon results of the comparing step;
generating, on a graphical user interface associated with the computerized system, a list of the plurality of requested tasks assigned to the at least one medical professional; and
updating the list of the plurality of requested tasks as the plurality of the requested tasks are completed.

13. The computerized method of claim 12, wherein the assigning step includes the steps of:
generating, on a graphical user interface associated with the computerized system, a list of available requested tasks for at least one medical institution professional based upon results of the comparing step; and
selectively assigning, using the graphical user interface, at least one of the available requested tasks to the medical institution professional from the list of available requested tasks.

14. The computerized method of claim 13, wherein the selectively assigning step is performed by the medical institution professional.

15. The computerized method of claim 13, wherein the selectively assigning step is performed by an individual other than the medical institution professional.

16. The computerized method of claim 13, wherein the list of available requested tasks are ordered by the computerized system based upon a priority algorithm.

17. The computerized method of claim 12, wherein the list of the plurality of requested tasks assigned to the at least one medical professional are ordered based upon a priority algorithm.

18. A computer assisted method for communicating medical reports to health care providers, comprising the steps of:
viewing by a communication operator, on a graphical user interface associated with a computerized system having access to a plurality of electronic medical reports, a present list of at least one of the plurality of electronic medical reports ready to be conveyed to a health care provider;
the communication operator contacting the health care provider;
the communication operator conveying information from the electronic medical report to the health care provider; and
logging by the communication operator, on the graphical user interface associated with the computerized system, information associated the contact with the health care provider.

19. The method of claim 18, wherein the electronic medical report includes one or more of the following:
a laboratory analysis result;
a radiology report;
a specialist physician's consultation report; and
a supervising physician's consultation report.

20. The method of claim 18, wherein if contacting step is not successful, then the method further includes the steps of:
noting, on the graphical user interface associated with the computerized system, a contact attempt by the communication operator; and
adding the electronic medical report, by the computerized system, to a subsequent list of electronic medical reports for later communication.

21. The method of claim 18 further comprising the step of, after or with the logging step, removing the electronic medial report from the present list of at least one of the plurality of electronic medical reports ready to be conveyed to a health care provider.

22. The method of claim 18 wherein the computerized system automatically affixes a time stamp to the information associated the contact with the health care provider.

23. A method for processing radiology cases comprising the steps of:
providing a software tool on a computer server accessible by a plurality of workstations coupled to the computer server over a computer network, wherein the software tool has access to a plurality of radiology case files corresponding to a plurality of pending radiology cases, and wherein the plurality of radiology case files includes information sufficient for one or more radiologists to conduct radiological examinations on the plurality of pending radiology cases;
assigning, with the assistance of the software tool, one or more of the plurality of pending radiology cases to a first radiologist;
accessing the software tool over the computer network by the first radiologist utilizing a first one of a plurality of workstations to view one or more radiology case files pertaining to a pending radiology case assigned to the first radiologist, and recording an examination of the pending radiology case assigned to the first radiologist in the software tool by the first radiologist utilizing the first one of the plurality of workstations;
removing, by the software tool, the pending radiology case assigned to the first radiologist from the plurality of pending radiology cases;
permanently recording by the software tool, electronic information pertaining to the assigning step and the recording step, the electronic information including a date and a time associated with the performance of at least the recording step.

24. The method of claim 23, further comprising the steps of:
communicating the recorded examination to a referring physician; and
storing a record of the communicating step, by or with the assistance of the software tool, the record of the communicating step including a date and a time associated with the performance of the communicating step.

25. The method of claim 24, wherein
the communicating step includes the step of accessing, with the assistance of the software tool and utilizing a second one of the plurality of workstations, the recorded examination by a communication assistant responsible for communicating data from the recorded examinations to the referring physician; and
the step of storing a record of the communication step includes a step of recording by the communication assistant, with the assistance of the software tool and utilizing the second one of the plurality of workstations, a record of communications with the referring physician.

26. The method of claim 23, wherein the assigning step includes a step of accessing, with the assistance of the software tool and utilizing a second one of the plurality of workstations, a graphical user interface that provides list of the plurality of pending radiology cases by a second individual, and utilizing the graphical user interface to assign from the list one or more of the plurality of pending radiology cases to the first radiologist.

27. The method of claim 26, wherein the list is ordered on the graphical user interface according to a priority algorithm.

28. The method of claim 23, further comprising the step of:
accessing, with the assistance of the software tool and utilizing a second one of the plurality of workstations, the recorded examination by a second radiologist;
logging by the second radiologist, with the assistance of the software tool and utilizing the second one of the plurality of workstations, comments associated with the recorded examination; and
permanently recording by the software tool, electronic information pertaining to accessing and logging steps, the electronic information including a date and a time associated with the performance of at least the logging step.
